(19) United States
(12) Patent Application Publication
(10) Pub. No.: US 2016/0000458 A1
(43) Pub. Date: Jan. 7, 2016

(54) PRECISION INCISION DEVICE AND METHOD

(71) Applicant: Beth Lynn COWAN, Berkeley, CA (US)

(72) Inventor: Beth Lynn Cowan, Berkeley, CA (US)

(21) Appl. No.: 14/766,140

(22) PCT Filed: Feb. 12, 2014
(86) PCT No.: PCT/US14/16125

§ 371 Date: Aug. 6, 2015

Related U.S. Application Data
(60) Provisional application No. 61/763,917, filed on Feb. 12, 2013.

(51) Int. Cl. A61B 17/3209; A61B 17/08

(52) U.S. Cl. CPC A61B 17/32093; A61B 17/08; A61B 17/085; A61B 2017/081; A61B 2017/320052

(57) ABSTRACT

A precision incision device, suitable for use on a human patient during surgery, comprises, in an embodiment, a pair of rails on which a cutting mechanism is slidably positioned. The rails are adhesively affixed to the body for at least the duration of the surgery. Maintained within the cutting mechanism is a blade or similar incising device. The blade is maintained in either of two positions: a retracted position, such that no sharp surface is exposed to a user, or an incising position, when the blade extends down beyond the rail to cause an incision in the patient. Following completion of the surgical procedure, a closure cover is affixed to the pair of rails to close the incision without the need for foreign bodies such as staples or stitches. In an alternative embodiment, only a single rail is required.

### RELATED APPLICATION

This application claims the benefit of U.S. Patent Application Ser. No. 61/763,917, filed Feb. 12, 2013, having the same title and inventor as the present application, and which is incorporated by reference herein in full.

### FIELD OF THE INVENTION

The present invention relates generally to medical devices and methods, and more particular relates to surgical devices and methods for causing rapid incisions of pre-set depth, and, in at least some embodiments, rapid closure of the incision.

### BACKGROUND OF THE INVENTION

For centuries, surgical incisions have typically been made by scalpels wielded by skilled physicians. Over that period, the importance of a skillful incision has become more and more clear, not only in the performance of the surgery requiring the incision, but also in the healing process the patient endures following the surgery.

In some surgeries, for example emergency Caesarean Sections, time is of the essence to protect the health of the newborn. Time is also of the essence for health and other reasons in many other surgeries. At the same time, the depth of the incision can be critical to avoiding unnecessary trauma to either the patient or, in the case of delivery, the newborn.

In the traditional method, the surgery site is prepped, and the surgeon manually controls a scalpel to create the desired incision. For a Caesarean Section, a low transverse incision is typical, in which an initial horizontal incision is made through the skin and abdominal wall one or two centimeters above the pubic symphysis. The tissues and muscles above the uterus are then cut through, and a further horizontal incision is made through the uterus, ultimately allowing extraction of the newborn.

Following delivery, in the traditional method the process is somewhat reversed. The incisions through the uterus, muscles and other tissue are closed, typically by dissolving stitches. Lastly, the skin and abdominal wall are closed by various means, typically either stitches or staples.

In terms of appearance after healing, as well as speed, the incision through the abdominal wall may be the most important. In the traditional approach, it is as desirable as it is difficult to create incisions in the skin that are nicely perpendicular to the skin, without “wobble” that causes skin flaps and leads to lumpy, uneven scars. It is also important that such incisions are limited in depth, to avoid unnecessary blood loss or other complications, which could be caused by, for example, incising epigastric vessels. While surgeons are typically highly skilled, it is inevitable that some incisions made by a hand-held scalpel will be less perpendicular, less straight, less or more deep, and thus fundamentally less precise than others. In addition, the manual application of stitches typically involves uneven pressure along the incision, creating lumps and bumps of scar tissue as the tissues reattach and heal.

For the above reasons, as well as numerous others, there has been a long-felt need for an incision device which substantially reduces, if not eliminates, the shortcomings associated with manually-created incisions and manual wound closures.

### SUMMARY OF THE INVENTION

The present invention provides a surgical device and method that enables a user such as a surgeon to create, reliably and repeatably, a surgical incision that is substantially perpendicular to the skin being incised, without the wobble or skin flaps typical of the manual approach. Further, in at least some embodiments, the device and method enable a substantially improved closure, where the skin on either side of the incision is returned to substantially the same point of contact as existed prior to the incision. As a result, healing is improved and the creation of unnecessary scar tissue is minimized.

The precision incision device of the present invention includes a cutting member (e.g., a scalpel blade) maintained within a housing member. In an embodiment, the housing member fits onto one or more guiding members, (e.g., tracks), which are affixed to the skin with an adherent for suitable for use in surgical procedures. In an embodiment, the guiding members comprise two parallel rails that are at least the length desired for the incision. In an embodiment, the rails have a hollow interior portion extending longitudinally for the length of the rail. In an alternate embodiment, the guiding members comprise hollow tubes, in some instances having a bottom surface configured to be securely affixed to the patient's. The guiding members, whether rails, tracks, tubes, or other embodiment, can be straight, curved, or flexible, and can be comprised of any suitable material, for example a hard plastic or equivalent, or, alternatively, a plastic that is sufficiently flexible in the longitudinal direction to follow the contour of the patient's body, while offering sufficient rigidity to maintain the cutting member in a position substantially perpendicular to the skin during the incising process.

The guiding member(s) can be affixed to the skin in a linear path, circular path, angular path, etc., depending on the desired incision. The guiding members can be metered (i.e., have measurements marked out along its length) to allow the user to precisely incise the skin and then to realign the skin during closure, as necessary.

In certain embodiments, the guiding member(s) are scored or segmented to allow the incision to be stretched, which results in the guiding members splitting apart into a plurality of interlocking components or segments. Once the stretching is completed, the interlocking components realign or snap back into the original configuration. The scoring can be created by perpendicular cuts (creating circular cross-sections), angular cuts (creating elliptical cross-sections), v-shaped cuts, etc. The interlocking components of the guiding members can also fit together in an overlapping configuration where the first component slides over the second component, the second component slides over the third component, etc. The scoring may run throughout the length of the guiding members or for only a portion of their length. The scoring may be present at regular or irregular intervals.

In certain embodiments, the scored guiding members can include flexible tubing, or its equivalent, within their hollow interiors. The flexible tubing may be made of silicon, rubber, elastic, or an equivalent, which, due to its elastic properties, can assist in realigning the guiding members into their original configuration. In other embodiments, the guiding members return to their original configuration without the assistance of flexible tubing within their hollow interiors. In certain embodiments, the flexible tubing directly adheres to the skin for maximum flexibility.

In certain embodiments, the hollow interiors of the guiding members contain antimicrobial fluid (e.g., betadine). When the incision is stretched and the interlocking components of the guiding members separate at the scoring, the antimicrobial fluid is released into the incision to maintain sterility. In certain embodiments the antimicrobial may be included in the adhesive affixing the device to the skin.

In an embodiment, closure of the wound in a manner which mates the edges of the incision is accomplished through the use of a closing member configured to slide over the guiding members once the surgical procedure is completed. The cutting member is removed, and the closing member is affixed to the guiding members so that the guide members are aligned relative to one another, and affixed to one another a distance which enables the skin to reattach without sutures. The closing member can be affixed at the top of the guide members, or can extend through the hollow interior of the guide members, or otherwise affix the guide members to one another in a manner which facilitates closure of the wound in a manner which avoids the lumpiness and scarring typically associated with manual suturing. The closing member may be made of plastic or any other material sufficient to maintain the guide members proximate to one another during the early stages of the healing process.

These and other features and aspects of various embodiments of the invention can be better appreciated from the following details description of the invention, taken together with the appended Figures.

### THE FIGURES

FIG. 1 depicts in perspective view an embodiment of a precision incision device comprising a pair of guide rails together with a cutting mechanism in accordance with the present invention.

FIG. 2A illustrates the precision incision device of FIG. 1 in place on a patient for a Pfannenstiel, or lower transverse, incision.

FIG. 2B illustrates the precision incision device of FIG. 1 in place on a patient for a vertical incision.

FIG. 3A illustrates an embodiment of a cutting member in accordance with the present invention.

FIG. 3B illustrates the adjustment mechanism of the cutting member of FIG. 3A.

FIG. 3C illustrates an alternative embodiment to the cutting member of FIG. 3A.

FIG. 4A illustrates the blade portion of the cutting member relative to a patient's skin, with the guide portion retracted.

FIG. 4B illustrates the blade portion of the cutting member with a guide portion as positioned during incising.

FIG. 5 illustrates the guide rails of FIG. 1 with the segments separated.

FIG. 6A shows a cover member to assist with closure.

FIG. 6B shows the cover member partially in position on the guide rails.

FIG. 7 shows an embodiment of a monorail version of the precision incision device of the present invention.

### DETAILED DESCRIPTION OF THE INVENTION

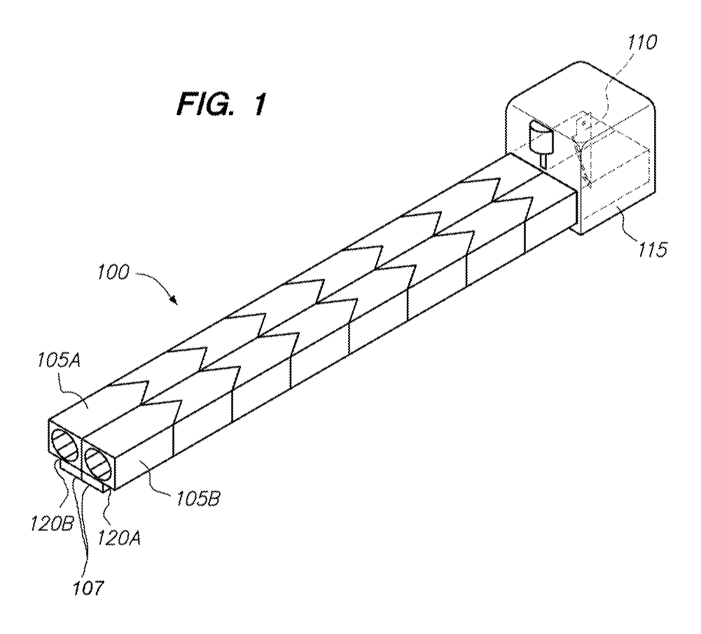

Referring first to FIG. 1, an embodiment of the precision incision device 100 of the present invention comprises includes a pair of guide rails 105A-105B. In cross-section, the guide rails, or tracks, can be generally rectangular, circular, oval, or other suitable shape. In at least some embodiments, the bottom surface includes a substantially flat portion 107 which permits the guide rails to be cemented to a patient at a surgical site. Such surgical adhesives are well known to those skilled in the art, and include, for example, acrylic adhesives, silicone-based adhesives, and hydrocolloid adhesives. In some embodiments and the adhesive selected, the adhesive is applied along the entirety of the rails, while in other embodiments the adhesive may be applied at intervals. The bottom surface portion 107 is preferably elastic and can be comprised of, for example, silicone or similar elastomers. In an embodiment, the rails are maintained slightly apart with only sufficient space between to permit a blade 110 to pass through, or can be joined with a thin membrane that can be cut concurrently with the incision.

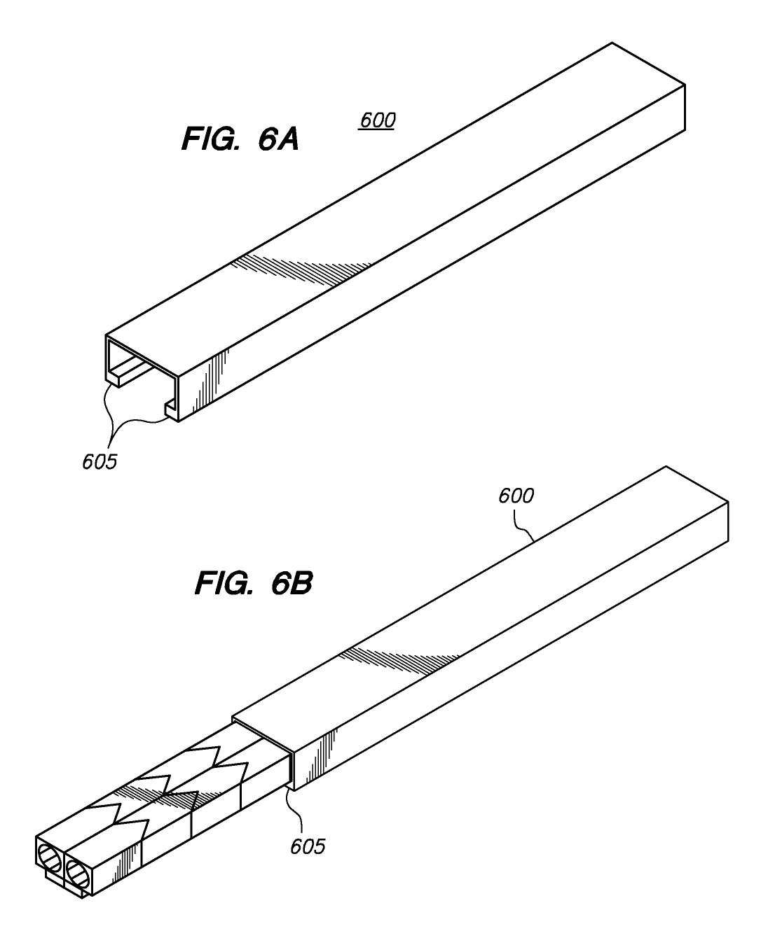

The blade 110 is maintained within a cutting member 115 which is mounted onto the rails 105A-105B and slides along their length. In an embodiment, the guide rails are substantially the same length as the intended incision, although the rails may be somewhat longer or shorter than the intended incision in some instances. The rails are preferably made of plastic or similar material which is suitably flexible to conform to the shape of body, while sufficiently rigid that the cutting member can readily slide along the rails. In an embodiment, during placement of the device 100 on the patient, a cover as shown in FIG. 6A can be maintained on the rails to keep them in position and properly aligned with one another.

In the embodiment shown in FIG. 1, a slight indentation 120A-120B exists at the lower edge of outer side of each of the rails to permit the cutting member 115 to be retained on the rails as it slides along them. However, in other embodiments, the indentation can be on the sides of the rails, or can be eliminated entirely. Further, for guide rails having a round or oval cross-section, the cutting member can be shaped to conform to the sides of the rails, eliminating the need for an indentation even for those embodiments where the cutting member cannot be lifted straight off the rails. In addition, each of the guide rails can have a longitudinal orifice 125A-125B extending therethrough. In an alternative embodiment, the guiding members comprise hollow tubes, in some instances having a bottom surface configured to be securely affixed to the patient's skin.

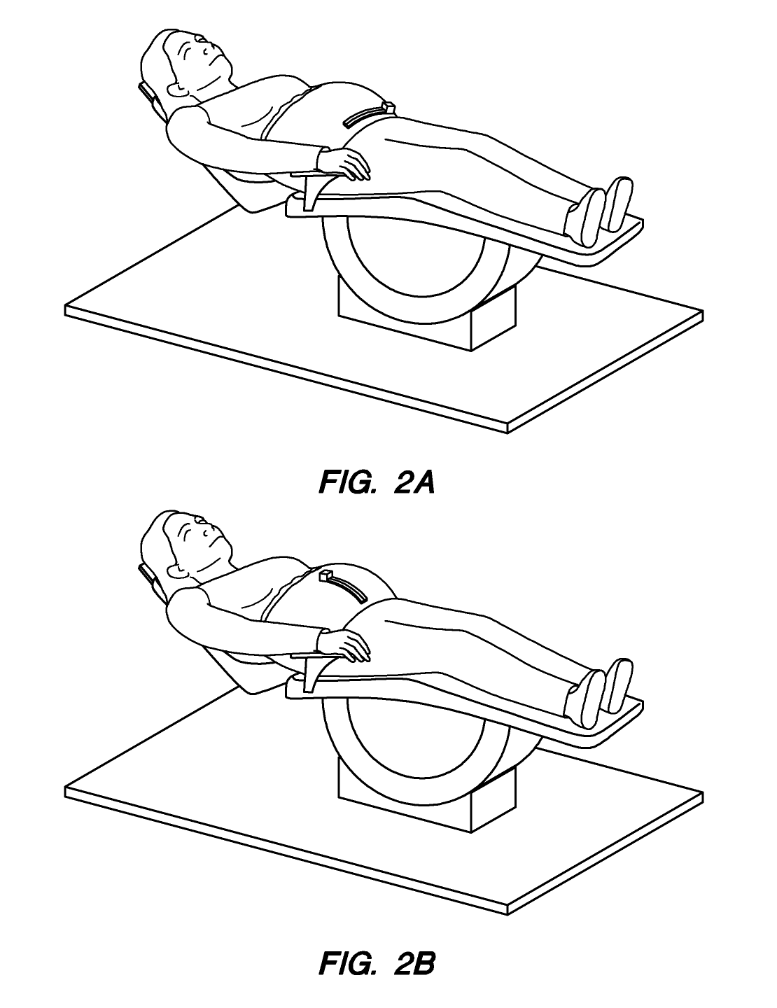

The guide rails, which can also be tracks, tubes, or other embodiment, can be straight, curved, or flexible, and can be comprised of any suitable material, for example a hard plastic or equivalent, or, alternatively, a plastic that is sufficiently flexible in the longitudinal direction to follow the contour of the patient's body, while offering sufficient rigidity to maintain the cutting member in a position substantially perpendicular to the skin during the incising process. As a result, the guide rails can be affixed to the patient as shown in FIGS. 2A and 2B. FIG. 2A shows the precision incision device 100 affixed to a patient for a Pfannenstiel incision, characteristic of a Caesarean section, while FIG. 2B shows an embodiment of the present invention arranged to create a vertical incision such as might be used in connection with abdominal wounds. It will be appreciated by those skilled in the art that, while reference to incisions in the skin is used herein for illustration, the present invention can be used for many types of surgery, including fascia, muscle, and so on.

The guide rails can be metered (i.e., have measurements marked out along its length) to allow the user to precisely incise the skin and then to realign the skin during closure, as necessary.

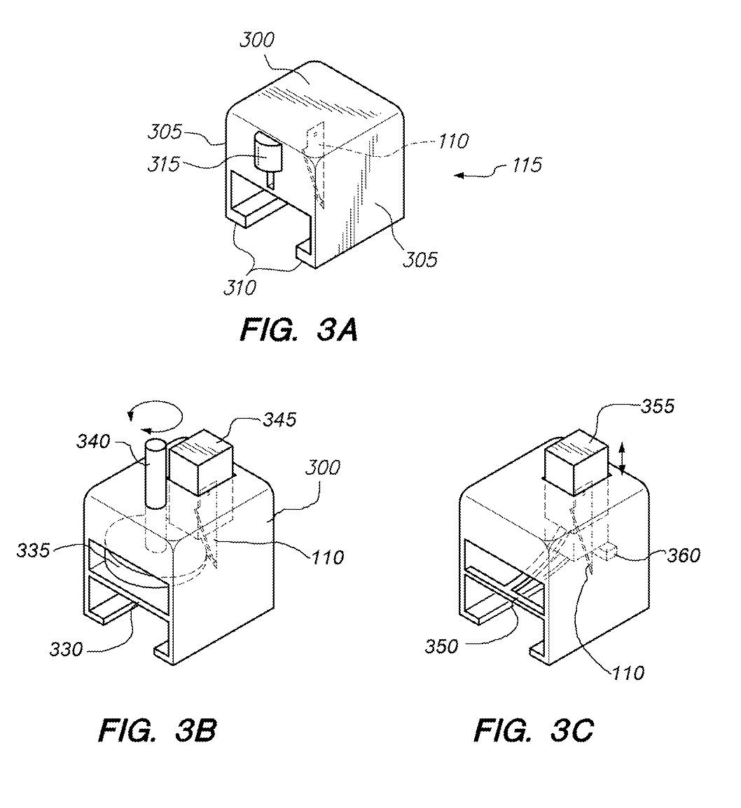

Referring next to FIG. 3A, an embodiment of the cutting member 115 can be better appreciated. The housing 300 of the cutting member 115 at least partially encloses the blade 110. A pair of side walls 305 extend downward on either side to engage the guide rails. For those embodiments having indentations in the guide rails for retention of the housing, lateral extrusions 310 can be provided on the side walls to mate with the indentations. As noted above, it will be appreciated by those skilled in the art that the indentations and mating extrusions are not required in all embodiments. The housing 300 can be made of metal, hard plastic, or an equivalent. The housing can be fitted to the cutting member or larger than the cutting member for ease of grip, and can be any suitable shape (e.g., rectangular, cylindrical, spherical, or ergonomically designed to fit the hand, etc.) convenient for manipulation by the surgical team. The cutting mechanism is disposable in some embodiments.

Referring next to FIGS. 3B-3C, further aspects of the invention can be better understood. In some surgeries, it is important that the incision through the skin is not too deep, to avoid nicking or cutting blood vessels and other tissues unnecessarily. In some embodiments it is desirable that the depth of cut can be adjusted in either specific increments or as desired by the surgeon. It will be appreciated by those skilled in the art that the thickness of skin varies depending upon the location on the body. Abdominal skin is typically between two and three millimeters thick. The skin on hands and feet is typically about 1.5 millimeters thick. As a result, it is desirable to provide the surgeon with the ability to pre-set the depth of the incision. This is accomplished with the cutter mechanism shown in FIG. 3A as follows: The cutting member or blade 110 can be directly affixed to the housing 300, or can be affixed to a spring-loaded clamping mechanism 315 which can be adjusted to set the depth of the incision.

If the height of the blade is adjustable, the adjustment can be performed by any suitable means, including a lever, a dial, a spring-loaded internal attachment, etc. The blade 110 can also be connected to the housing by one or more longitudinal connecting members, which permits the blade to move up and down with finger pressure. In such an embodiment, mechanical stops can to provided to permit adjust of the depth of the incision. The positioning member can, in some embodiments, include safety devices to prevent accidental lowering of the cutting member. The positioning member is configured to lower or raise the cutting member by a precise amount (e.g., to a depth of about 1.7 mm) to create an incision that is directly perpendicular to the skin surface.

In certain embodiments where the blade 110 is rigidly affixed to the housing 300, various implementations of the housing/blade combination can be provided, where the blade is pre-positioned to provide different incision depths, and the surgeon selects the combination that gives the depth desired for a particular incision. This approach eliminates certain costs associated with the adjustment mechanism of other embodiments of the invention.

For the embodiment shown in FIG. 3B, the housing 300 includes a stop plate 330 on which is mounted a rotary wedge 335 connected to a rotary knob 340. Rotation of the rotary wedge adjusts the depth of the incision by providing a mechanical stop. Depressing a spring-loaded button 345 moves the blade down past the cutter mechanism, between the rails, and into the skin until the bottom of the button contacts the rotary wedge, to create an incision of the prescribed depth. Releasing the button causes the spring (not shown for clarity but supported inside the housing and maintained inside the button) to retract the blade into the housing 300. The blade can be any suitable surgical blade sized to permit full retraction while also causing the desired incision, for example a titanium blade of size/shape 11, or 15, or other suitable shape and size. For some types of incisions, a blunt tip will be desirable.

In certain alternate embodiments, the housing member may include attachments in conjunction with, or as a substitute for, the cutting member in order to aid in hemostasis during incision. Examples include a harmonics attachment, a laser attachment, or a coagulation attachment.

In a further alternative embodiment, shown in FIG. 3C, the housing 300 supports a flexure 350, to which a standard blade 110 is attached. A button 355 permits the surgeon to cause the blade to extend below the cutter and through the rails to the skin of the patient. Depressing the button through its full stroke causes (depending upon the embodiment) either the flexure 350 or the bottom of the button 355 to contact a mechanical stop 360 on the inside of the housing 300, the vertical position of which sets the maximum depth of the incision. It will be appreciated that the position of the mechanical stop can be fixed during manufacture, or can be adjusted, although the least expensive assembly will typically preset the position of the stop during manufacture, with a selection of cutters providing the surgeon with a variety of incision depths, as determined by the surgeon to be appropriate for a particular patient. The flexure 350 is configured in an upward arcuate shape to provide a spring force, such that it flattens when the button is depressed, and forces the blade upward into the housing when the button is released. The combination of the flexure, button, and mechanical stop can be sized to ensure that the tip of the blade is not exposed to users when the blade is in the retracted position.

As discussed above, in at least some embodiments, the blade is maintained within the housing both prior to depressing the button 355 and following retraction, thus exposing the hospital staff to no sharps during the ordinary handling of the cutter mechanism. To prevent the button from being pressed inadvertently, and thus exposing the blade, a safety lock 365 can be inserted through the housing 300 between the stop 360 and the flexure 350.

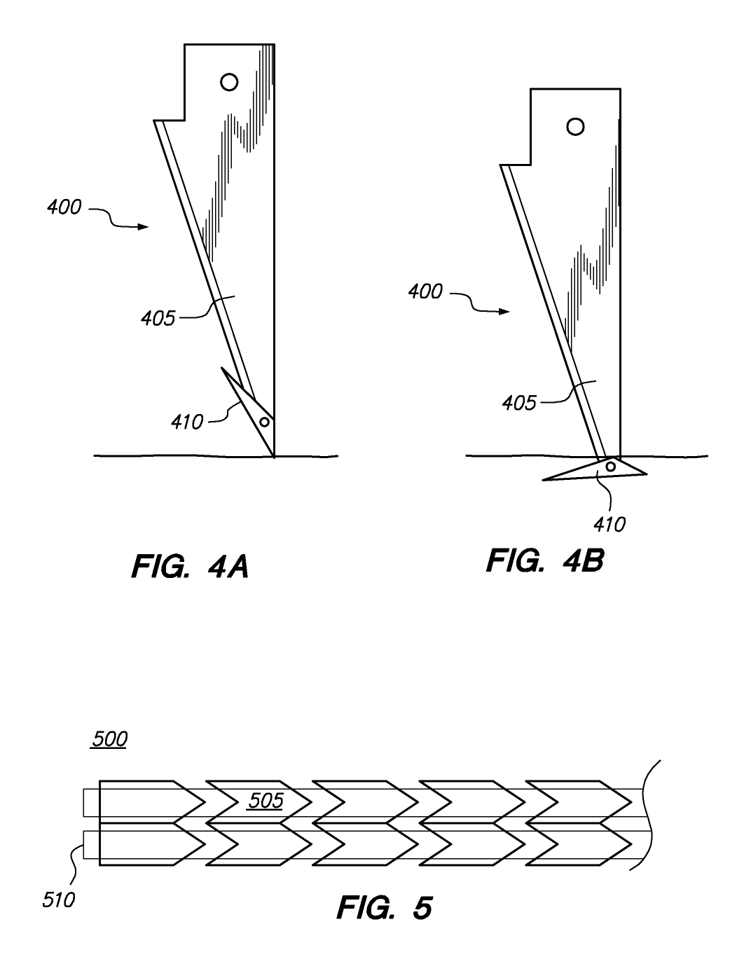

Referring next to FIGS. 4A-4B, a still further aspect of the present invention, used in some embodiments, can be better appreciated. In particular, the cutting member or blade 400 can include a first longitudinal portion 405 having a cutting edge and a second portion 410 without a cutting edge. The second portion 410 is smaller than the first portion 405 and pivotably affixed to the tip of the first portion 405 such that the portion 410 is fitted parallel to and against tip of the first portion during insertion of the blade 400 into the skin. Once the cutting member has been inserted into the skin, the second portion, or skin guide, flips perpendicular to the first portion and the cutting member slightly retracts (e.g., by about 0.2 mm). This allows the skin guide second portion to be placed at the interface between the skin layer and the subcutaneous layer and to slightly elevate the skin layer. As the housing member containing the cutting member moves along the guiding members, the second portion remains in the interface between the skin layer and the subcutaneous layer and adjusts the depth of the cutting member to assist the surgeon in not making an incision beyond the skin layer while also allowing the surgeon to compensate for small variations in the thickness of the skin layer. In such embodiments, the mechanical stop of the cutter mechanisms shown in FIGS. 3A-3C can be made slightly lower to allow a deeper initial puncture that permits the skin guide to be properly rotated into place.

Once the incision is complete, the second portion collapses, for example by a slight backward motion along the incision, back into a parallel configuration with the first portion for retraction of the cutting member from the skin. In certain embodiments, the second portion is made of a bioabsorbable material (e.g., Vicryl or another polymer) such that all or part of the second portion may detach and remain in the interface between the skin layer and the subcutaneous layer during retraction of the cutting member from the skin.

As noted above, in an embodiment of a method in accordance with the invention, the user affixes the guide rails onto the patient's skin at the desired location for incision using an adherent. In certain embodiments, the adherent is also infused with antimicrobial fluid so that the incision area remains sterile. The user then fits the housing member containing the cutting member onto the guiding members. The user lowers the cutting member out from the housing member using the positioning member, as discussed above, such that the cutting member is inserted into the skin up to the desired incision depth. The user then slides the housing member, with the cutting member lowered, along the guiding member until the desired incision length is executed. When the incision has been executed, the cutting member is retracted back into the housing member.

Referring next to FIG. 5, in some embodiments, the guide rails 500 are scored or segmented to allow the incision to be stretched, which, for example, results in the guiding members splitting apart into a plurality of interlocking components or segments 505 as the skin is stretched, for example during delivery of a newborn. In some embodiments, elastic tubes 510 can be maintained within the hollow interior of the segments to assist in returning the segments to an interlocked position following the procedure, although in some embodiments the elasticity of the skin is sufficient to return the segments to a fully interconnected position without the next for the elastic tubes.

Once the surgical procedure is complete, there is typically a need to close the incision. At this point, a still further aspect of the present invention can be appreciated. As discussed above, the guide rails are affixed to the skin, and, in some embodiments, spaced very closely together. The cutting member and guide rails have cooperated to cause an incision that is perpendicular and without the variation associated with manual incising. As a result, when the guide rails are returned to their initial position following the procedure, the edges of the incised skin match along the entirety of the incision. By affixing the guide rails together, the wound is closed without the need for stitches, staples, or other foreign bodies that can cause infection, keloid formation, and increased scarring, thus allowing improved wound healing and decreased scarring. FIGS. 6A-6B show an embodiment of a closure cover 600 which slides along guide rails, with extrusions 605 similar to the extrusions 310 on the cutting member. Like the cutting member, the closure cover 600 is retained on the rails by the indentations 120A-B. Once the wound has healed sufficiently, the guide rails are removed.

As an alternative to the cover 600, a plurality of clips can be used which extend over the two rails and holds them together. As a further alternative, an interlocking structure similar to the closure mechanism on a zip-lock bag can be used to close the incision, where the interlocking elements are positioned on the rails and opened when the incision is made, then closed either manually or by sliding the cutting member in the opposite direction and with the blade retracted.

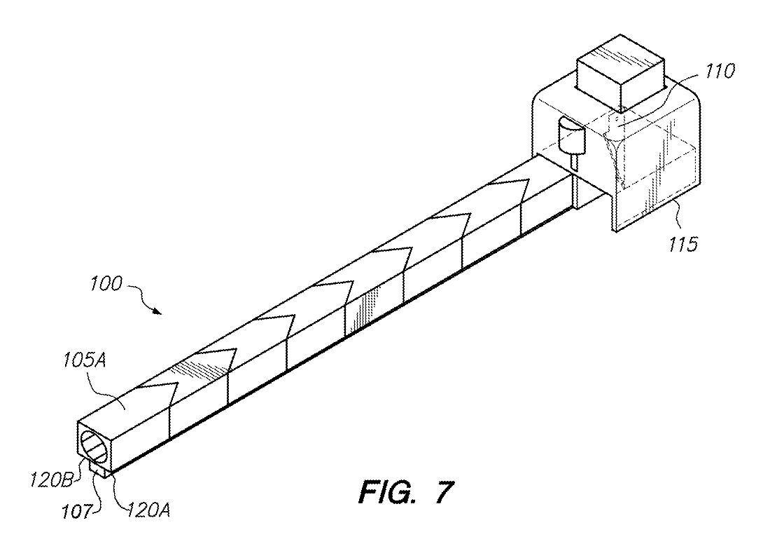

In a further alternative embodiment, FIG. 7 shows a single guide rail configured to support a cutting member. In particular, a single rail 700, substantially identical to a single rail of the pair shown in FIG. 1, is cemented to the skin in the same way as discussed in connection with FIG. 1. A reconfigured housing 705 slides along the rail and, like the cutters of FIGS. 3A-3C, maintains the blade in a retracted position until the button 710 is pushed, at which time the blade extends downward to the pre-selected depth. The blade is maintained at one side of the rail 700, with the housing suitably mated to the rail to permit a vertical cut. The embodiment of FIG. 7 does not provide the closure mechanism of the embodiment of FIG. 1, but does provide for a quick and precise incision.

From the foregoing, it will be appreciated that the present invention also lends itself to robotic or other remotely-controlled surgery, including establishing a structure and protocol by which robotic control of incisions is possible. In particular, the placement of the precision incision device of the present invention can be remotely controlled by qualified medical personnel, the depth of incision preset, and the incision completed with the present invention holding the skin in place for counter tension, and also providing depth control. Likewise, robotic creation of ports for laparoscopic surgery is made possible by the present invention.

From the teachings herein, those skilled in the art will appreciate that a precision incision device, including various alternatives, has been described which provides the benefits of: 1) protecting surgical personnel from accidental exposure to the cutting member, 2) preventing the unnecessary incision of the subcutaneous layer, 3) maintaining a perpendicular incision on the skin, thus reducing damage to the skin and the resulting healing time, 4) minimizing exposure of the surface areas of the incision to microbes, 5) ensuring precision in the length and shape of the incision, such as through use of metered guiding members, and 6) ensuring precision in the realigning and sealing of the incision, among other benefits. As will be understood by those familiar with the art, the invention may be embodied in other specific forms without departing from the spirit or essential characteristics thereof. Accordingly, the above disclosure of the present invention is intended to be illustrative and not limiting of the invention.

I claim:

1. An incision control device for use during surgical procedures comprising
a pair of rails conformable to a portion of the human body,
a cutting mechanism slidable along the pair of rails for creating an incision in the space between the rails, the cutting mechanism causing the incision to be substantially perpendicular to the body, and
a closure cover for maintaining the pair of rails adjacent to one another following completion of the surgical procedure to cause the edges of the incision to be repositioned adjacent one another in substantially the same position as before the surgical procedure.

2. An incision control device for use during surgical procedures comprising
at least one rail of a material suitable for conforming to a portion of the human body,
at least one flexible strip affixed to the at least one rail for receiving an adhesive for temporarily affixing the at least one rail to human skin,
a cutting mechanism slidable along the at least one rail,
a blade member mounted within the cutting mechanism, the blade member being maintainable in a first retracted position except during the creation of an incision, and in a second incising position during the creation of an incision, such that no sharp surface is exposed while the blade member is in the retracted position, and the blade member further being maintained substantially perpendicular to the body while in the second incising position.

3. The device of claim 2 wherein the at least one rail is a pair of rails.

4. The device of claim 3 further comprising
a closure cover for maintaining the pair of rails adjacent to one another following completion of the surgical procedure to cause the edges of the incision to be repositioned adjacent one another in substantially the same position as when the rails were affixed without the need for staples or stitches.

4. The device of claim 1 further comprising
a skin guide rotatably maintained substantially at the tip of the blade member such that the guide can be rotated into the interface between the skin and the subcutaneous layer.
